(19) United States
(12) Patent Application Publication
(10) Pub. No.: US 2013/0202723 A1
(43) Pub. Date: Aug. 8, 2013

(54) METHOD OF PRODUCING A CHINESE HERBAL COMPOSITION FOR TREATING TERMINAL CANCER PATIENTS WITH CONSTIPATION

(71) Applicants: Yu-Te Liu, Changhua City (TW); Jaung-Geng Lin, Changhua City (TW)

(72) Inventors: Yu-Te Liu, Changhua City (TW); Jaung-Geng Lin, Changhua City (TW)

(21) Appl. No.: 13/563,047

(22) Filed: Jul. 31, 2012

(30) Foreign Application Priority Data
Feb. 7, 2012 (TW) 101103841

(51) Int. Cl. A61K 36/484; A61P 1/10

(52) U.S. Cl. 424/757

(57) ABSTRACT

Constipation is a common and serious problem for terminal cancer patients, often resulting in deterioration of the overall condition of the patients and causing great anxiety in their family members. Terminal cancer patients, according to the principle of palliative care and the symptoms of pain, are usually given morphine-like analgesics orally or by injection to ease the pain, and are therefore very likely to suffer from constipation, poor appetite, bloating and other relevant symptoms. The Chinese herbal composition of rhubarb and licorice extracts presented in this study can notably improve the symptoms of constipation, poor appetite, and bloating, and these results are statistically significant.

### BACKGROUND OF THE INVENTION

1. Field of the Invention

The present invention relates to a Chinese herbal composition used for treating constipation, in particular, in terminal cancer patients with constipation.

2. Description of the Prior Art

In western medicine, constipation is a common problem found in palliative care. Constipation causes various gastrointestinal symptoms such as bloating, poor appetite, and pain, as well as poor quality of life. Deterioration of a patient's overall condition may also induce extreme anxiety in family members. In Taiwan, studies have shown that at least half of patients admitted to the hospital experience problems with defecation. For terminal cancer patients, according to the principles of palliative care, morphine-like analgesics are often given orally or by injection to ease pain. However, these drugs may increase ion and water adsorption in the gastrointestinal tract and reduce the defecation reflex, since the mechanism of morphine-like analgesics is to reduce smooth muscle motility and increase smooth muscle tension. This subsequently leads to an increased tension of the ileocecal valve and anal sphincter, as well as reduced large intestine and small intestine motility. Therefore, patients have a high probability of developing constipation. Additionally, if not treated properly, constipation may result in poor appetite and slower bowl movement. Once this cycle is formed, this problem may be even harder to treat.

Foreign statistics have indicated that 50-80% of the patients in palliative care units need to use laxatives to treat constipation. Certain patients depend on enemas to deal with their constipation for longer periods of time. For example, domestic data as disclosed in “Risk factors and clinical management of constipation for terminally ill cancer patients” by Mei-Feng Su in 2005 has suggested that the prevalence of constipation in terminal cancer patients was 58.9% and 83.3% according to subjective and objective assessments, respectively. As for clinical treatments of constipation, the majority are non-independent care measures, such as laxatives (92.4%) and enemas (67.9%). Only a handful are independent care measures, including health education (43.4%), abdominal massage (20.8%), and digging (11.3%). Hence, common clinical treatments of constipation are to prescribe laxatives and enemas. Common drugs used for treating constipation are listed in the table below:

| Type | Mechanism of action | Side effects | Representative drugs | Exclusion conditions |
|---|---|---|---|---|
| bulk-forming | Water absorption | flatulence, | Psylliym, | |
| laxatives | results in an increase | bloating, | Sterculia, | |
| | of the volume of the | esophageal or | Methylcellulose, | |
| | stool which | intestinal | Calcium | |
| | consequently | obstruction | polycarbophil | |
| | stimulate the bowl | | | |
| | movement | | | |
| osmotic laxatives | | | | |
| Saline | Increase intestinal | abdominal | Magnesium oxide, | Renal dysfunction, |
| laxatives | osmotic pressure by | cramping, | Magnesium sulfate, | chronic renal failure, |
| | osmosis and cause | dehydration, | Sodium sulfate, | and congestive heart |
| | water retention in the | magnesium | Sodium citrate | failure |
| | intestine | poisoning | | |
| Poorly | poorly absorbed | Flatulence, | Sorbitol, Lactulose | Patients with |
| absorbed | sugars catalyzed by | bloating | | Galactosemia |
| sugars | enteric bacteria will | | | and intestinal |
| | reduce the pH in the | | | obstruction |
| | intestine, which in | | | |
| | turn will stimulate | | | |
| | intestinal motility and | | | |
| | secretion | | | |
| glycerin | Produce a high | Stimulation of | | Children under |
| | osmotic pressure in | the rectum and | | two years of |
| | small intestine, retain | burning sensation | | age |
| | the water in the gut, | | | |
| | and stimulate | | | |
| | intestinal wall, | | | |
| | increase bowl | | | |
| | movement and | | | |
| | facilitate defecation | | | |
| Stimulant | Stimulate intestinal | Bloating, | Derivatives of | |
| laxatives | smooth muscle plexus | diarrhea, | Diphethlmethane | |
| | and increase intestinal | electrolyte | (e.g. Bisacodyl) and | |
| | motility or water/ | imbalance, | Anthraquinones (e.g. | |
| | electrolyte secretion | allergic reactions, | Sennoside, cascara | |
| | | and liver | segrada) and castor | |
| | | toxicity chronic | oil | |
| | | constipation, and | | |
| | | difficult | | |
| | | defecation | | |
| lubricants | Stick to and lubricate | Dehydration and | mineral oil | Before going |
| | stool and prevent | reduce the | | to bed and bed |
| | water adsorption in | absorption of | | rest patients |
| | the intestine from | fat-soluble | | |
| | feces to soften and | vitamins; cannot | | |
| | increase the volume of | be used with | | |
| | stool and facilitate | laxatives or in | | |
| | defecation | patients with | | |
| | | acute or chronic | | |
| | | lipid pneumonia | | |

According to the table shown above, bloating and diarrhea are the most common side effects of many clinical drugs, and most of these drugs are toxic. This results in numerous restrictions in their clinical applications, and are therefore not preferred treatments for terminal cancer patients in palliative care units. On the other hand, clinical results indicate that patients may refuse to use analgesics if they have serious concerns regarding chronic constipation, which inevitably causes more pain. In conclusion, clinical drugs are ineffective in improving constipation in terminal cancer patients. Moreover, they may increase medical-related costs.

In traditional Chinese medicine (TCM), constipation was considered a symptom caused by transduction dysfunction of the large intestine which resulted from various stagnations in intestinal junction, defecation, spleen, yin, and yang. In spite of the fact that the main lesion of constipation is in large intestine, the symptoms are usually correlated with liver, spleen, and kidney dysfunctions. Accordingly, with regard to the treatments of constipation, TCM often focused on restoring the function of the liver, spleen, kidney, and intestine.

Rhubarb (Rheum rhabarbarum) belongs to Polygonaceae perennials, and has thick petiole, small flowers, and triangular leaves. Its flower is greenish-white or rosy-red, and the leaves contain toxic oxalic acid with the minimum lethal dose (LDLo) around 600 mg/kg if taken orally. The roots of rhubarb are edible, but contain anthraquinones and thereby are not recommended for use in pregnant women. Rhubarb is usually collected in late fall before the stems or leaves are wilted or prior to germination in early spring. After removal of its fine roots and skin, rhubarb can be eaten directly after drying or used as a seasoning ingredient, along with alcohol, in food preparation. The pharmaceutical properties of rhubarb as disclosed in TCM are cold and bitter, and it mainly affects the spleen, stomach, large intestine, liver, and heart. The major effects of this herb include constipation relief, detoxification, improvement of circulation, removal of sputum, and fever relief.

Licorice (Glycyrrhiza uralensis) is a leguminous perennial with erect stems, cylindrical-shaped roots, singular pinnate leaves, and butterfly-shaped purple flowers that bloom in summer, and the fruit is an oblong pod. The roots and underground stems are edible. The pharmaceutical properties of licorice in TCM are sweet and calm, and mainly affect the heart, lung, spleen and stomach, with potential effects such as fever relief, detoxification, cough relief, and pain relief. However, long-term or high dose consumption of this herb may cause pseudo-hyperaldosteronism, edema, hypertension, arrhythmia, myocardial damage, and muscle weakness, etc. In general, licorice is often collected in spring and fall and, following removal of its fibrous roots, dried and cut into thick slices. It can be eaten directly or honey-roasted. Thus far, no studies using these two Chinese herbs to treat terminal cancer patients are reported.

Therefore, the inventor(s) of the present invention developed a novel Chinese herbal composition and a unique method of producing this novel composition for treating terminal cancer patients with constipation.

The inventor(s) presented a poster relating to this invention at the 11^{th} Australian Palliative Care Conference in Cairns from Tuesday 30 Aug. to Friday 2 Sep. 2011. The title of the poster is “Improving Constipation in Patients at Terminal Stage: A pilot study on the Efficacy of taking Herbal Formula: DaHuang GangCao Tang”.

### SUMMARY OF THE INVENTION

The present invention provides a Chinese herbal composition comprising rhubarb and licorice. This composition can significantly improve constipation in terminal cancer patients.

In one aspect, the present invention provides a Chinese herbal composition comprising of rhubarb extract and licorice extract.

In another aspect, the present invention reveals an alternative therapy for treating constipation in terminal cancer patients under palliative care.

The Chinese herbal composition as mentioned above contains rhubarb and licorice and has a weight ratio of 1:1 to 1:8, which is adjusted according to the patient's age, weight, physical condition, and severity of the disease. This composition is given 1 to 4 times a day, and is administered orally or transdermally, and dissolved in warm water and given in proportions so as to allow adequate adsorption in impaired gastrointestinal tract of the patients. This provides protection against poor appetite, nausea, vomiting, bloating, and constipation found in terminal cancer patients in palliative care units.

The herb mentioned in the present invention refers to a Chinese herb or Chinese herb extract, and the methods of extraction of the Chinese herbal composition of the present invention include production or extraction methods known in prior arts, such as water extraction, alcohol extraction, and concentration. In addition, the steps involved in the concentration process used for producing standardized dosing of the Chinese herb include extraction using water, separation, concentration, spray drying and molding, etc.

The Chinese herbal composition also contains an excipient or a medically acceptable carrier. The excipient is a diluent, filler, adhesive, disintegrating agent, or lubricant, and the carrier can be granules, powder, capsules, or solution. The composition can be mixed with the abovementioned excipients or carriers to make tablets, pills, creams, powder, solution, or capsules. In addition, the term “powder” as used herein refers to Chinese herbs ground into fine powder. The term “tablet” as used herein means fine powder of Chinese herbs mixed with carriers such as adhesives, lubricants, as well as lactose or corn starch, etc. The term “pill” as used herein is made by adding water, honey, or flour paste. The term “cream” as used herein is produced by extraction of the herb followed by concentration and then boiling to a semi-solid form. The term “solution” as used herein is obtained by slow cooking the herbs and subsequent filtration. The term “capsule”, on the other hand, refers to capsule carriers filled with finely ground Chinese herb, in which lactose or corn starch may be added as effective diluents.

### DETAILED DESCRIPTION OF THE PREFERRED EMBODIMENTS

The invention is explained in detail in the examples given below and should not be construed to limit the scope of invention.

The weight ratio of rhubarb to licorice of the composition tested in the present invention is 4:1. However, a weight ratio of 1:1, 2:1 or 8:1 are all acceptable to use in clinical trials. This trial is a randomized, single-blinded, pilot study. The participating subjects are terminal cancer patients admitted to the palliative care unit at Changhua Christian Hospital. A total of 27 patients were enrolled and divided into a test group and a control group for clinical trial.

The participating patients received a total of 2 grams of rhubarb and 0.5 g of licorice daily, in four doses, for 6 days. For feeding by NG tube or via surgical G- or J-tube, the herbs can be dissolved in water prior to feeding. The selected assessment tools include quality-monitoring index in the Palliative Care Pilot Plan, the European Organization for Research and Treatment of Cancer (EORTC QLQ-C30 V3.0), and the Eastern Cooperative Oncology Group Performance Status (ECOG) assessment forms.

The EORTC QLQ-C30 V3.0 was developed by the European Organization for Research and Treatment to assess the quality of life of cancer patients, and includes five functional subscales (physical, role, cognitive, emotional, and social functions), three symptom subscales (fatigue, pain, nausea/vomiting), and the general health status subscale is composed of the abovementioned subscales in various combination.

On the other hand, the Eastern Cooperative Oncology Group Performance Status (ECOG) is for assessing the daily performance status (PS) of the patients. At present, the ECOG developed life quality assessment form is the most commonly used clinical tool. The clinical symptoms are assigned a score from 0 to 4, wherein 0 means asymptomatic, 1 refers to limited symptoms with no effects on daily life, 2 and 3 indicate bed rest time being less and more than 50%, respectively, and 4 indicates long-term bed rest.

The subjects participated in the present study are divided into a test group and a control group (see Table 1), and were tested based on the medication regimen as indicated above. The Mix model was used to analyze the data collected from two groups and within the same group at different time points. The results indicated that the symptoms of constipation, loss of appetite and bloating in terminal cancer patients between two groups, with or without using the said composition, are significantly different (p<0.05), while no significant differences between the two groups were found for nausea and vomiting. Additionally, comparison analysis performed within the individual group suggested no major differences in nausea and vomiting, loss of appetite, bloating or constipation.

TABLE 1

| Test group | | Control group | |
|---|---|---|---|
| (n = 12) | | (n = 15) | |
| n | M ± SD | n | M ± SD |

Variables

| | | |
|---|---|---|
| Male/Female | 7/5 | 10/5 |
| Age (yrs) | | |
| Mean ± standard deviation | 53.68 ± 3.36 | 59.68 ± 4.56 |
| range | 38-71 | 28-90 |

Education

| | | |
|---|---|---|
| None | 3 | 5 |
| Elementary school | 5 | 4 |
| Middle school | 4 | 5 |
| High school | 0 | 0 |
| College | 0 | 1 |

Religion

| | | |
|---|---|---|
| None | 6 | 8 |
| Taoism | 4 | 5 |
| Buddhism | 2 | 2 |

Use of NG tube

| | | |
|---|---|---|
| Yes | 6 | 8 |
| No | 6 | 7 |

Use of laxatives

| | | |
|---|---|---|
| Yes | 2 | 14 |
| No | 10 | 1 |

Use of analgesics

| | | |
|---|---|---|
| Yes | 7 | 5 |
| No | 5 | 10 |

Terminal cancer patients may not be able to eat normally or they may loss their appetite. Therefore, nutrition intake may be affected. The corresponding treatment in western medicine is usually placing a NG tube to maintain normal feeding. Thus, this study excluded two groups of patients who have had an NG tube placed, and analyzed and compared the remaining data using the Mixed model. The results showed significant differences in comparison of constipation, loss of appetite, and bloating between two groups (p<0.05), whereas no significant differences were observed in nausea/vomiting (p>0.05). As shown in Table 2, this result is consistent with the findings obtained from studies including patients with a NG tube. Thus, NG tube has no effects on the results of the study.

TABLE 2
Analysis of the parameters of gastrointestinal symptoms
(exclusion of NG tube)

between
groups

| Variables | F | P |
|---|---|---|
| Constipation | 4.74 | 0.013* |
| Loss of appetite | 4.481 | 0.016* |
| Bloating | 7.488 | 0.002* |
| Nausea/vomiting | 1.819 | 0.175 |

As shown in Table 3, without exclusion of the patients with nasogastric (NG) tube, after analysis of the average constipation scores, the pre-test scores, mid-test scores, post-test scores, and improvement scores of the test group and the control group are 2.17±0.22, 1.29±0.22, 1±10.24, 53.91%, and 1.67±0.26, 1.67±0.25, 1.77±0.28, −5.99%, respectively

After analysis of the loss of appetite scores, the pre-test scores, mid-test scores, post-test scores, and improvement scores of the test group and the control group are 1.92±0.34, 1.13±0.28, 0.75±0.23, 60.93%, and 1.5±0.29, 1.53±0.32, 1.58±0.353, −5.33%, respectively.

As for bloating, the pre-test scores, mid-test scores, post-test scores, and improvement scores of the test group and the control group after analysis are 2.33±0.22, 1.71±0.23, 1.08±0.28, 53.64%, and 1.5±0.263, 1.73±0.21, 1.85±0.25, −23.33%, respectively.

TABLE 3

| | | |
|---|---|---|
| | | between |
| Test group | Control group | groups |

| | | | | |
|---|---|---|---|---|
| Variables | M ± SD | M ± SD | F | P |

Constipation

| | | | | |
|---|---|---|---|---|
| Pre-test | 2.17 ± 0.22 | 1.67 ± 0.26 | 4.746 | 0.013* |
| Mid-test | 1.29 ± 0.22 | 1.67 ± 0.25 | | |
| Post-test | 1 ± 0.24 | 1.77 ± 0.28 | | |

Loss of appetite

| | | | | |
|---|---|---|---|---|
| Pre-test | 1.92 ± 0.34 | 1.5 ± 0.29 | 4.424 | 0.017* |
| Mid-test | 1.13 ± 0.28 | 1.53 ± 0.32 | | |
| Post-test | 0.75 ± 0.23 | 1.58 ± 0.35 | | |

Bloating

| | | | | |
|---|---|---|---|---|
| Pre-test | 2.33 ± 0.22 | 1.5 ± 0.263 | 7.328 | 0.002* |
| Mid-test | 1.71 ± 0.23 | 1.73 ± 0.21 | | |
| Post-test | 1.08 ± 0.28 | 1.85 ± 0.25 | | |

Nausea/vomiting

| | | | | |
|---|---|---|---|---|
| Pre-test | 0.79 ± 0.29 | 0.27 ± 0.15 | 1.809 | 0.177 |
| Mid-test | 0.04 ± 0.04 | 0 | | |
| Post-test | 0.21 ± 0.11 | 0.08 ± 0.08 | | |

Value are mean ± SD.;
*p < 0.05

Based on the above results, symptoms such as constipation, poor appetite and bloating were dramatically improved after oral administration of the Chinese herbal composition produced in the present invention. In addition to constipation relief, patient appetite and bloating were also improved and sleeping problems was also diminished.

The purpose of the present invention is to treat constipation found in terminal cancer patients. Terminal cancer patients are cancer patients who do not response to anti-cancer cure therapy, are experiencing relapses, are not suitable for or refused to receive anti-cancer treatments. Moreover, terminal cancer patients' quality of life are usually decreased due to poor physical condition and impaired immune response caused by tumor, surgery, chemotherapy, or radiotherapy, and common symptoms may include pain, fatigue, nausea, vomiting, dry mouth, loss of appetite, constipation, difficulty breathing, insomnia, and depression. Morphine-like analgesics or patches, tricyclic antidepressants, diuretics, iron, and other drugs are usually given in order to relieve these symptoms. In particular, a significant portion of these patients require morphine-like analgesics to ease the pain, which inevitably aggravates the severity of constipation. However, western medicine, such as laxatives, have been shown to have poor effects in treating constipation. Therefore, the purpose of the present invention is to combine western medicine and traditional Chinese medicine and develop a novel treatment for constipation which may relieve the gastrointestinal discomfort caused by surgery, chemotherapy/radiotherapy, and alleviate symptoms, including dry mouth, oral ulcer, constipation, and difficulty in opening the mouth which occur during treatment. Accordingly, the patients can complete the treatments successfully and receive the maximal benefits of these western therapies. As for serious signs of illness, such as weight loss, loss of appetite, listlessness, as well as cancer pain, traditional Chinese medicine may improve the quality of life of cancer patients through physical conditioning.

The abovementioned description and specific examples, while indicating embodiments of the invention, represent one of the feasible applications of the present invention only, since various changes and modifications within the spirit and scope of the invention will become apparent to those skilled in the art. One skilled in the art, based upon the description herein, may utilize the present invention to its full extent. It should be understood; therefore, that the invention is not limited to the preferred embodiments shown.

What is claimed is:

1. A Chinese herbal composition comprising rhubarb and licorice.

2. The Chinese herbal composition of claim 1, wherein the composition is for treating terminal cancer patients with constipation.

3. The Chinese herbal composition of claim 1, wherein the composition is administered orally.

4. The Chinese herbal composition of claim 1, wherein the weight ratio of rhubarb to licorice is 1:1, 2:1, 4:1, and 8:1.

5. The Chinese herbal composition of claim 1, wherein the composition is produced by water extraction.

6. The Chinese herbal composition of claim 1, wherein the composition is produced by alcohol extraction.

7. The Chinese herbal composition of claim 4, wherein the composition is produced by subsequent concentration following extractions.

8. The Chinese herbal composition of claim 5, wherein the composition is produced by subsequent concentration following extractions.

9. The Chinese herbal composition of claim 1, wherein the composition contains a medically acceptable carrier.

10. The Chinese herbal composition of claim 1, wherein the carriers used in the composition are diluents, filler, adhesives, disintegrating agents, or lubricants.

11. The Chinese herbal composition of claim 1, wherein the composition is produced as tablets, pills, creams, powder, solutions, or capsules.

12. The method of using the Chinese herbal composition of claim 1, to treat terminal cancer patients, wherein the composition is administered orally.

13. The method of claim 12, wherein the therapy is for treating constipation caused by administration of morphine-like analgesics, tricyclic antidepressants, diuretics, and iron.

14. The method of claim 12, wherein the composition is administered 1 to 4 times a day.
